(19) United States
(12) Patent Application Publication
(10) Pub. No.: US 2024/0173376 A1
(43) Pub. Date: May 30, 2024

(54) TREM-1 INHIBITORS FOR THE TREATMENT OF MARFAN SYNDROME

(71) Applicants: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); UNIVERSITÄT BERN, BERN (CH); Sorbonne Université, PARIS (FR); Université Paris Cité, PARIS (FR)

(72) Inventors: Hafid AIT-OUFELLA, PARIS (FR); Christoph MUELLER, BERN (CH); Marie VANDESTIENNE, BONN (DE)

(21) Appl. No.: 18/552,054

(22) PCT Filed: Mar. 11, 2022
(86) PCT No.: PCT/EP2022/056400

§ 371 Date: Sep. 22, 2023

(30) Foreign Application Priority Data
Mar. 12, 2021 (EP) 21305307.7

(51) Int. Cl. A61K 38/08; A61K 38/10; A61K 39/00; C07K 16/28

(52) U.S. Cl. CPC A61K 38/08; A61K 38/10; C07K 16/2803; A61K 2039/505

(57) ABSTRACT

Marfan Syndrome is a pathology responsible for a high morbidity and mortality. Apart from surgery, treatment options are limited. It is therefore essential to develop new pharmacological approaches to limit aortic dilatation and/or rupture. Now, the inventors have demonstrated a critical role for TREM-1 in the pathophysiology of ascending aortopathy related to Marfan disease, In particular, the inventors showed that i) TREM-1 is expressed by macrophages in the aortic wall of Marfan patients, ii) TREM-1 is expressed by macrophages in the aortic wall of Marfan mice, and iii) TREM-1 deficiency protects against aortic rupture in a mouse model of Marfan syndrome. Blocking TREM-1 could therefore be a therapeutic approach for Marfan-related aortopathy.

### FIELD OF THE INVENTION

The present invention is in the field of medicine, in particular vascular diseases.

### BACKGROUND OF THE INVENTION

Marfan syndrome is caused by mutations in the FBN1 gene (15q21) that codes for Fibrillin-1, an essential connective tissue protein. Border forms are secondary to mutations in the TGFBR2 gene located on chromosome 3, which codes for TGF-beta receptor. Its prevalence is estimated at 1/5000, i.e. 12,000 patients in France. Transmission is autosomal dominant. Thus, the disease affects both genders indiscriminately and an affected person has a 50% risk of transmission of the mutation. Symptoms can appear at any age and vary greatly from one person to another, even within the same family. Skeletal signs are often warning signs and may include dolichostenomelia (excessive length of the extremities), tall stature, arachnodactyly, joint hypermobility, etc. Ophthalmologic damage includes axial myopia which can lead to retinal detachment and displacement of the lens (ectopy or dislocation, a characteristic sign). There may also be skin signs (stretch marks), a risk of pneumothorax, and dural ectasia. More importantly, it is the cardiovascular disorders that conditions the prognosis of patients with Marfan syndrome with progressive dilatation of the ascending aorta accompanied by a high risk of a potentially fatal aortic dissection. Mitral valve (prolapse) or aortic valve abnormalities of the bicuspid type are also described. Pregnancy increases the risk of complications and should therefore be carefully monitored (Keane M G, Pyeritz, Circulation, 2008). Much progress has been made (Pyeritz et al, Heart 2009) in the management of Marfan patients but morbi-mortality remains too high.

The only treatment now recommended by experts is a beta-blocker, propranolol, which limits aortic dilatation and the risk of dissection (Shores et al, New Engl J Med 1994; Ladouceur et al, Am J Cardiol 2007). This treatment is recommended upon confirmation of the diagnosis of Marfan syndrome or related syndrome in case of aortic dilatation, or without aortic dilatation from the age of 4 years in the presence of a mutation. Medical treatment should be continued even after heart surgery and during pregnancy. It should not be stopped after childbirth (Omnes et al, Int J Gynaecol Obstet. 2013). In women with Marfan syndrome pregnancy is associated with an over-risk of aortic dissection. Above 45 mm aortic diameter, pregnancy is contraindicated. Type 1 angiotensin receptor blockers have been evaluated but their beneficial impact remains controversial. In an animal model of Marfan's disease, Losartan, an AT1R antagonist, improves arterial wall architecture and reduces aortic dilatation (Hibashi et al, Science, 2006). On the other hand, the interest of this molecule in humans is less clear. A randomized, double-blind, multicenter study reported no benefit (Milleron et al, Eur Heart J) while another study with a comparable methodology (N=192) reported a benefit of Losartan on the progression of aortic dilatation (Mullen et al. Lancet 2019). Surgical (+/− endovascular) treatment of the ascending aorta, alone or associated with a procedure on the aortic valve, is considered when the diameter of the ascending aorta is greater than 50 mm or when the increase in dilatation is rapid (more than 3 mm in one year, verified by 2 techniques), (adapted from the recommendations of the European Society of Cardiology 2014 (Erbel et al, Eur Heart J 2014). Surgical procedures are associated with significant perioperative complications such as leakage or dissection on the distal portion of the anastomosis.

In summary, Marfan Syndrome is a pathology responsible for a high morbidity and mortality. Apart from surgery, treatment options are limited. It is therefore essential to develop new pharmacological approaches to limit aortic dilatation and/or rupture.

The pathophysiological mechanisms causing aortic dilatation and dissection in Marfan syndrome are not clearly understood. Few pathophysiological work has evaluated the involvement of the immuno-inflammatory response in Marfan syndrome. However, several elements argue for a pathogenic role of immunity in the progression and complications of the disease. First of all, in the blood of Marfan patients, the level of M-CSF is higher in those with high aortic dilatation rates. In aortic media and aortic adventitia, there is a significant increase in T-cell and CD68+ macrophage infiltration in Marfan patients compared to aorta of control subjects. (Radonic et al PlosOne 2012) (D'amico, Int J Mol Sci. 2020; He, J Thorac Cardiovasc Surg 2006). In mouse models mimicking Marfan syndrome, there is also infiltration of inflammatory cells into the aortic wall with local overexpression of genes encoding chemokines (CCL-2, CCL-5), chemokine receptors (CX3CR1) and cytokines (IL-1b). However, the mechanisms that regulate the recruitment and activation of these macrophages are unknown, as is their involvement in vascular disease.

TREM-1 is a receptor expressed by monocytes/macrophages and neutrophils discovered in 2000. TREM-1 has mainly been studied during infectious states where it works closely with TLRs to amplify the inflammatory response (Bouchon et al, Nature 2001). TREM-1 is associated, via its transmembrane domain, with an adaptor protein DAP12. This association triggers the activation of signaling pathways leading to the mobilization of calcium reserves, a rearrangement of the actin cytoskeleton and the activation of transcriptional factors such as NF-kB. This cascade induces the production of metalloproteases, pro-inflammatory cytokines, chemokines and neutrophil degranulation (Arts et al. J leukoc biol 2013). Our team has recently identified a major role of TREM-1 in post-infarction cardiac remodeling (Boufenzer et al, Circ Res 2015) and in the development of atherosclerosis (Joffre et al, JACC 2016) by modulating various myeloid cell functions, migration, activation, cytokine production and lipid endocytosis. It has been recently shown that TREM-1 expression is induced by angiotensin II via its AT1R receptor (Vandestienne et al, JCI 2020). TREM-1 is also involved in the pathophysiology of degenerative abdominal aortic aneurysm (Vandestienne et al, JCI 2020). However the role of TREM-1 in Marfan Syndrome has not yet been investigated.

### SUMMARY OF THE INVENTION

The present invention is defined by the claims. In particular, the present invention relates to the use of TREM-1 inhibitors for the treatment of Marfan Syndrome.

### DETAILED DESCRIPTION OF THE INVENTION

The first object of the present invention relates to a method of treating Marfan Syndrome in a patient in need thereof comprising administering a therapeutically effective amount of a TREM-1 inhibitor.

As used herein, the term “Marfan Syndrome” has its general meaning in the art and refers to a systemic disease of connective tissue characterized by a variable combination of cardiovascular, musculo-skeletal, ophthalmic and pulmonary manifestations. Symptoms can appear at any age and vary greatly between individuals even within the same family. Cardiovascular involvement is characterized by 1) progressive dilation of the aorta accompanied by an increased risk of aortic dissection, which affects prognosis; the aortic dilation can result in a leaky aortic valve; and 2) mitral insufficiency, which can be complicated by arythmias, endocarditis or cardiac insufficiency. Skeletal involvement is often the first sign of the disease and can include dolichostenomelia (excessive length of extremities), large size, arachnodactyly, joint hypermobility, scoliotic deformations, acetabulum protrusion, thoracic deformity (pectus carinatum or pectus excavatum), dolichocephaly of the anteroposterior axis, micrognathism or malar hypoplasia. Ophthalmic involvement results in axile myopia, which can lead to retinal detachment and lens displacement (ectopia or luxation are characteristic signs). Ocular complications, particularly lens ectopia, can lead to blindness. Cutaneous signs (vergetures), a risk of pneumothorax and dural ectasia can also occur. In the vast majority of cases, Marfan syndrome is caused by mutations of the FBN1 gene (15q21), which codes for fibrilline-1, a protein essential for connective tissues. Frontier forms have been identified that are secondary to mutations in the TGFBR2 gene located on chromosome 3, which codes for a TGF-beta receptor.

As used herein, the term “treatment” or “treat” refers to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder (such as dilation of the aorta and/or aortic dissection), or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In particular embodiment, the TREM-1 inhibitor prevent or treat the dilation of the aorta in the patient affected with Marfan Syndrome.

Thus, in particular embodiment, the TREM-1 inhibitor prevent an aortic dissection in the patient affected with Marfan Syndrome.

In particular, the TREM-1 inhibitor of the present invention is particularly suitable for preventing ascending aorta rupture.

As used herein, the term “TREM-1” has its general meaning in the art and refers to the Triggering receptor expressed on myeloid cells 1. TREM-1 is a member of the Ig-superfamily, the expression of which is up-regulated on phagocytic cells in the presence of bacteria or fungi (Bouchon A et al. Nature 2001; 230:1103-7). An exemplary amino acid sequence is represented by SEQ ID NO: 1. It was previously described that TREM-1 can be shed or secreted from the membrane of activated phagocytes and can be found in a soluble form in body fluids. Accordingly, the term “sTREM-1” refers to the soluble form of the human TREM-1 receptor.

>sp|Q9NP99|TREM1_HUMAN Triggering
receptor expressed on myeloid cells 1
OS = Homo sapiens OX = 9606
GN = TREM1 PE = 1 SV = 1
SEQ ID NO: 1
MRKTRLWGLLWMLFVSELRAATKLTEEKYE
LKEGQTLDVKCDYTLEKFASSQKAWQIIRD
GEMPKTLACTERPSKNSHPVQVGRIILEDY
HDHGLLRVRMVNLQVEDSGLYQCVIYQPPK
EPHMLFDRIRLVVTKGFSGTPGSNENSTQN
VYKIPPTTTKALCPLYTSPRTVTQAPPKST
ADVSTPDSEINLTNVTDIIRVPVFNIVILL
AGGFLSKSLVFSVLFAVTLRSFVP

As used herein, the term “TREM-1 inhibitor” refers to any compound, chemical, antibody, or peptide, naturally occurring or synthetic, that directly or indirectly decreases the activity and/or expression of TREM-1. Functionally conservative variations of known TREM-1 inhibitors are also intended to be covered by this description. This includes, for example only, deuterated variations of known inhibitors, inhibitors comprising non-naturally occurring amino-acids, functional variations of peptide inhibitors involving a different sequence of amino acids, inhibitors created by codon variations which code for the same amino-acid sequence of a known inhibitor or functional variation thereof, versions of peptides described herein in which one or more of the amino acids can be, individually, D or L isomers. The invention also includes combinations of L-isoforms with D-isoforms.

Common TREM-1 inhibitors include peptides which may be derived from TREM-1, or TREM-like-transcript-1 (“TLT-1”). Any peptide which competitively binds TREM-1 ligands, thereby reducing TREM-1 activity and/or expression is a TREM-1 inhibitor. These peptides may be referred to as “decoy receptors.”

In some embodiments, the TREM-1 inhibitor is a peptide that is disclosed in WO2014037565. Examples of such peptides are listed below in Table A. LR17 is a known, naturally occurring direct inhibitor of TREM-1 which functions by binding and trapping TREM-1 ligand. LR12 is a 12 amino-acid peptide derived from LR17. LR12 is composed of the N-terminal 12 amino-acids from LR17. Research suggests that LR12 is an equivalent TREM-1 inhibitor when compared to LR17. LR6-1, LR6-2 and LR6-3 are all 6 amino-acids peptides derived from LR17. These peptides may function in the same manner as LR12.

TABLE A
Different peptides that can be
used as TREM-1 inhibitors

| Peptide | | |
|---|---|---|
| name | Sequence | SEQ ID |
| LR17 | LQEEDAGEYG | SEQ ID NO: 2 |
| | CMVDGAR | |
| LR12 | LQEEDAGEYG | SEQ ID NO: 3 |
| | CM | |
| LR6-1 | LQEEDA | SEQ ID NO: 4 |
| LR6-2 | EDAGEY | SEQ ID NO: 5 |
| LR6-3 | GEYGCM | SEQ ID NO: 6 |
| LP17 | LQVEDSGLYQ | SEQ ID NO: 7 |
| | CVIQHPP | |
| LP12 | LQVEDSGLYQ | SEQ ID NO: 8 |
| | CV | |
| LP6-1 | LQVEDS | SEQ ID NO: 9 |
| LP6-2 | EDSGLY | SEQ ID NO: 10 |
| LP6-3 | GLYQCV | SEQ ID NO: 11 |

In some embodiments, the TREM-1 inhibitor is a peptide derived from TLT-1 or TREM-1, in particular peptides as described herein.

In some embodiments, the TREM-1 inhibitor is a short TLT-1 peptide consisting of less than 50 amino acids, preferably consisting of between 6 and 20 amino acids, more preferably consisting of between 6 and 17 amino acids, wherein said TLT-1 peptide comprises between 6 and 20 consecutive amino acids from the human TLT-1 having an amino acid sequence as set forth in SEQ ID NO: 12 (MGLTLLLLLLLGLEGQGIVGSLPEVLQAPVGSSILVQCHYRLQDVKAQKVWCRFLPE GCQPLVSSAVDRRAPAGRRTFLTDLGGGLLQVEMVTLQEEDAGEYGCMVDGARGP QILHRVSLNILPPEEEEETHKIGSLAENAFSDPAGSANPLEPSQDEKSIPLIWGAVLLVG LLVAAVVLFAVMAKRKQGNRLGVCGRFLSSRVSGMNPSSVVHHVSDSGPAAELPLD VPHIRLDSPPSFDNTTYTSLPLDSPSGKPSLPAPSSLPPLPPKVLVCSKPVTYATVIFPGG NKGGGTSCGPAQNPPNNQTPSS); or a sequence having at least 60, 65, 70, 75, 80, 85 or 90% identity with the amino acid sequence as set forth in SEQ ID NO: 12; or a function-conservative variant or derivative thereof.

In some embodiments, the TREM-1 inhibitor is a TLT-1 peptide consisting of 6 to 12, 13, 14, 15, 16, 17, 18, 19 or 20 amino acids and comprising an amino acid sequence as set forth in SEQ ID NO: 2, SEQ ID NO: 3, SEQ ID NO: 4, SEQ ID NO: 5 or SEQ ID NO: 6. or a sequence having at least 60, 65, 70, 75, 80, 85 or 90% identity with the amino acid sequence as set forth in SEQ ID NO: 2, SEQ ID NO: 3, SEQ ID NO: 4, SEQ ID NO: 5, or SEQ ID NO: 6, respectively; or a function-conservative variant or derivative thereof.

In some embodiments, the TREM-1 inhibitor is a TLT-1 peptide comprising or consisting of an amino acid sequence as set forth in SEQ ID NO: 2, SEQ ID NO: 3, SEQ ID NO: 4, SEQ ID NO: 5, or SEQ ID NO: 6; or a sequence having at least 60, 65, 70, 75, 80, 85 or 90% identity with the amino acid sequence as set forth in SEQ ID NO: 2, SEQ ID NO: 3, SEQ ID NO: 4, SEQ ID NO: 5, or SEQ ID NO: 6, respectively; or a function-conservative variant or derivative thereof.

In some embodiments, the TREM-1 inhibitor is a TLT-1 peptide having an amino acid sequence as set forth in SEQ ID NO: 2, SEQ ID NO: 3, SEQ ID NO: 4, SEQ ID NO: 5, or SEQ ID NO: 6; or a sequence having at least 60, 65, 70, 75, 80, 85 or 90% identity with the amino acid sequence as set forth in SEQ ID NO: 2, SEQ ID NO: 3, SEQ ID NO: 4, SEQ ID NO: 5, or SEQ ID NO: 6, respectively; or a function-conservative variant or derivative thereof.

In some embodiments, the TREM-1 inhibitor is a TLT-1 peptide having an amino acid sequence as set forth in SEQ ID NO: 3, also known as LR12; or a sequence having at least 60, 65, 70, 75, 80, 85 or 90% identity with the amino acid sequence as set forth in SEQ ID NO: 3; or; or a function-conservative variant or derivative of SEQ ID NO: 3.

In some embodiments, the TREM-1 inhibitor is a short TREM-1 peptide consisting of less than 50 amino acids, preferably consisting of between 6 and 20 amino acids, more preferably consisting of between 6 and 17 amino acids, wherein said TREM-1 peptide comprises between 6 and 20 consecutive amino acids from the human TREM-1 having an amino acid sequence as set forth in SEQ ID NO: 1 or a function-conservative variant or derivative thereof.

In some embodiments, the TREM-1 inhibitor is a TREM-1 peptide consisting of 6 to 12, 13, 14, 15, 16, 17, 18, 19 or 20 amino acids and comprising an amino acid sequence as set forth in SEQ ID NO: 7, SEQ ID NO: 8, SEQ ID NO: 9, SEQ ID NO: 10 or SEQ ID NO: 11; or a sequence having at least 60, 65, 70, 75, 80, 85 or 90% identity with the amino acid sequence as set forth in SEQ ID NO: 7, SEQ ID NO: 8, SEQ ID NO: 9, SEQ ID NO: 10 or SEQ ID NO: 11, respectively; or a function-conservative variant or derivative thereof.

In some embodiments, the TREM-1 inhibitor is a TREM-1 peptide comprising or consisting of an amino acid sequence as set forth in SEQ ID NO: 7, SEQ ID NO: 8, SEQ ID NO: 9, SEQ ID NO: 10 or SEQ ID NO: 11; or a sequence having at least 60, 65, 70, 75, 80, 85 or 90% identity with the amino acid sequence as set forth in SEQ ID NO: 7, SEQ ID NO: 8, SEQ ID NO: 9, SEQ ID NO: 10 or SEQ ID NO: 11, respectively; or a function-conservative variant or derivative thereof.

In some embodiments, the TREM-1 inhibitor is a TREM-1 peptide having an amino acid sequence as set forth in SEQ ID NO: 7, SEQ ID NO: 8, SEQ ID NO: 9, SEQ ID NO: 10 or SEQ ID NO: 11 or a sequence having at least 60, 65, 70, 75, 80, 85 or 90% identity with the amino acid sequence as set forth in SEQ ID NO: 7, SEQ ID NO: 8, SEQ ID NO: 9, SEQ ID NO: 10 or SEQ ID NO: 11, respectively; or a function-conservative variant or derivative thereof.

As used herein, the term “identity” or “identical”, when used in a relationship between the sequences of two or more peptides, refers to the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. \2, 387 (1984); Genetics Computer Group, University of Wisconsin, Madison, Wis.), BLASTP, BLASTN, and FASTA (Altschul et al., J. MoI. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

As used herein, the term “function-conservative variants” denotes peptides derived from the peptides as described herein, in which a given amino acid residue in a peptide has been changed without altering the overall conformation and function of said peptides, including, but not limited to, replacement of an amino acid with one having similar properties (such as, for example, similar polarity, similar hydrogen bonding potential, acidic or basic amino acid replaced by another acidic or basic amino acid, hydrophobic amino acid replaced by another hydrophobic amino acid, aromatic amino acid replaced by another aromatic amino acid). It is commonly known that amino acids other than those indicated as conserved may differ in a peptide so that the percent of amino acid sequence similarity between any two peptides of similar function may vary and may be, for example, from 70% to 99% as determined according to an alignment method such as by the Cluster Method, wherein similarity is based on the MEGALIGN algorithm. A “function-conservative variant” also includes peptides which have at least 20%, 30%, 40%, 50%, or 60% amino acid identity with the peptides as described herein, for example as determined by BLAST or FASTA algorithms, and which have the same or substantially similar properties or functions as the peptides as described herein. Preferably “function-conservative variants” include peptides which have at least 60%, 65%, 70%, 75%, 80%, 85% or 90% amino acid identity with the peptides as described herein and which have the same or substantially similar properties or functions as the peptides as described hereinabove. As used herein, the term “derivative” refers to a variation of a peptide or of a function-conservative variant thereof that is otherwise modified in order to alter the in vitro or in vivo conformation, activity, specificity, efficacy or stability of the peptide. For example, said variation may encompass modification by covalent attachment of any type of molecule to the peptide or by addition of chemical compound(s) to any of the amino-acids of the peptide. In some embodiments, the peptide or function-conservative variants or derivatives thereof as described hereinabove may have D- or L-configuration. In some embodiments, the amino acid from the amino end of the peptide or function-conservative variant or derivative thereof as described hereinabove has an acetylated terminal amino group, and the amino acid from the carboxyl end has an amidated terminal carboxy group. In addition, the peptide or function-conservative variant or derivative thereof as described hereinabove may undergo reversible chemical modifications in order to increase its bioavailability (including stability and fat solubility) and its ability to pass the blood-brain barrier and epithelial tissue. Examples of such reversible chemical modifications include esterification of the carboxy groups of glutamic and aspartic amino acids with an alcohol, thereby removing the negative charge of the amino acid and increasing its hydrophobicity. This esterification is reversible, as the ester link formed is recognized by intracellular esterases which hydrolyze it, restoring the charge to the aspartic and glutamic residues. The net effect is an accumulation of intracellular peptide, as the internalized, de-esterified peptide cannot cross the cell membrane. Another example of such reversible chemical modifications includes the addition of a further peptide sequence, which allows the increase of the membrane permeability, such as a TAT peptide or Penetratin peptide (see—Charge-Dependent Translocation of the Trojan. A Molecular View on the Interaction of the Trojan Peptide Penetratin with the 15 Polar Interface of Lipid Bilayers. Biophysical Journal, Volume 87, Issue 1, 1 Jul. 2004, Pages 332-343).

The peptides or function-conservative variants or derivatives thereof as described hereinabove may be obtained through conventional methods of solid-phase chemical peptide synthesis, following Fmoc and/or Boc-based methodology (see Pennington, M. W. and Dunn, B. N. (1994). Peptide synthesis protocols. Humana Press, Totowa.). Alternatively, the peptides or function-conservative variants or derivatives as described hereinabove may be obtained through conventional methods based on recombinant DNA technology, e.g., through a method that, in brief, includes inserting the nucleic acid sequence coding for the peptide into an appropriate plasmid or vector, transforming competent cells for said plasmid or vector, and growing said cells under conditions that allow the expression of the peptide and, if desired, isolating and (optionally) purifying the peptide through conventional means known to experts in these matters or eukaryotic cells that express the peptide. A review of the principles of recombinant DNA technology may be found, for example, in the text book entitled “Principles of Gene Manipulation: An Introduction to Genetic Engineering,” R. W. Old & S. B. Primrose, published by Blackwell Scientific Publications, 4th Edition (1989).

Additional examples of TREM-1 inhibitors include those disclosed by patent application WO 2015018936. These include, but are not limited to, antibodies directed to TREM-1 and/or sTREM-1 or TREM-1 and/or sTREM-1 ligand, small molecules inhibiting the function, activity or expression of TREM-1, peptides inhibiting the function, activity or expression of TREM-1, siRNAs directed to TREM-1, shRNAs directed to TREM-1, antisense oligonucleotide directed to TREM-1, ribozymes directed to TREM-1 and aptamers which bind to and inhibit TREM-1. Antibodies have been shown to inhibit TREM-1 as well. Representative antibodies are described, for example, in U.S. Publication No. 20130309239 and U.S. Pat. No. 9,000,127. Additional examples of TREM-1 inhibitors also include those disclosed in WO2011 047097. As described in U.S. patent publications 20090081199 and 20030165875, fusion proteins between human IgG1 constant region and the extracellular domain of mouse TREM-1 or that of human TREM-1 can be used, as a decoy receptor, to inhibit TREM-1. Another TREM-1 inhibitor is TLT-1, as disclosed in Washington, et al., “A TREM family member, TLT-1, is found exclusively in the alpha-granules of megakaryocytes and platelets,” Blood. 2004 Aug. 15; 104(4):1042-7. Additional TREM-1 inhibitors include MicroRNA 294, which has been shown to target TREM-1 by dual-luciferase assay activity. Naturally-occurring TREM-1 inhibitors include curcumin and diferuloylmethane, a yellow pigment present in turmeric. Inhibition of TREM-1 by curcumin is oxidant independent. Accordingly, curcumin and synthetic curcumin analogs, such as those described in U.S. Publication Nos. 20150087937, 20150072984, 20150011494, 20130190256; 20130156705, 20130296527, 20130224229, 20110229555; and 20030153512; U.S. Pat. Nos. 7,947,687, 8,609,723, and PCT WO 2003105751.

As used herein, the term “therapeutically effective amount” refers to a sufficient amount of the TREM-1 inhibitor to treat Marfan Syndrome in the subject. It will be understood, however, that the total daily usage of the agent is decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific agent; and like factors well known in the medical arts. For example, it is well within the skill of the art to start doses of the compound at levels lower than those required to achieve the desired therapeutic effect and to gradually increase the dosage until the desired effect is achieved. However, the daily dosage of the agent may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the agent for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the inhibitor of the present invention is combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Sterile injectable solutions are prepared by incorporating the active ingredient at the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The invention will be further illustrated by the following figures and examples. However, these examples and figures should not be interpreted in any way as limiting the scope of the present invention.

### FIGURES

FIG. 1: qPCR quantification of Trem-1 mRNA in the aorta of control (CTR) or Marfan mice (mgR/mgR) at 2 months (Left, N=10/group) and 6 months (Right, N=5-6/group) of age. *, P<0.05, **, P<0.01.

FIG. 2. Flow cytometry validation of the lack of expression of TREM-1 by CD45+CD11b+ myeloid cells in mgR/mgR/Trem-1−/− mice.

FIG. 3: Survival curves of Marfan mgR/mgR/Trem-1^{+/+} mice and littermate mice deficient in TREM-1 mgR/mgR/Trem-1^{-/-}. N=26-28/group, ***, P=0.004

FIG. 4: Survival curves of Marfan mgR/mgR mice treated either with isotype (N=15) or with anti-TREM-1 stimulating antibody IP (0.1 mg/kg 2 times/week, 16-0621-86, clone MEL-14) (N=15).

### EXAMPLE

### Results:

### TREM-1 is Expressed by Macrophages in the Aortic Wall of Marfan Patients.

First, we evaluated the expression of TREM-1 by immunofluorescent labeling on surgical specimens from patients with Marfan syndrome and compared to normal aorta (organ donors). We showed that TREM-1 is not detected in normal aorta. In contrast, TREM-1 is expressed in the wall of the ascending aorta of Marfan patients, in the media, and this expression is (largely) colocalized with CD68+ macrophages.

### TREM-1 is Expressed by Macrophages in the Aortic Wall of Marfan Mice.

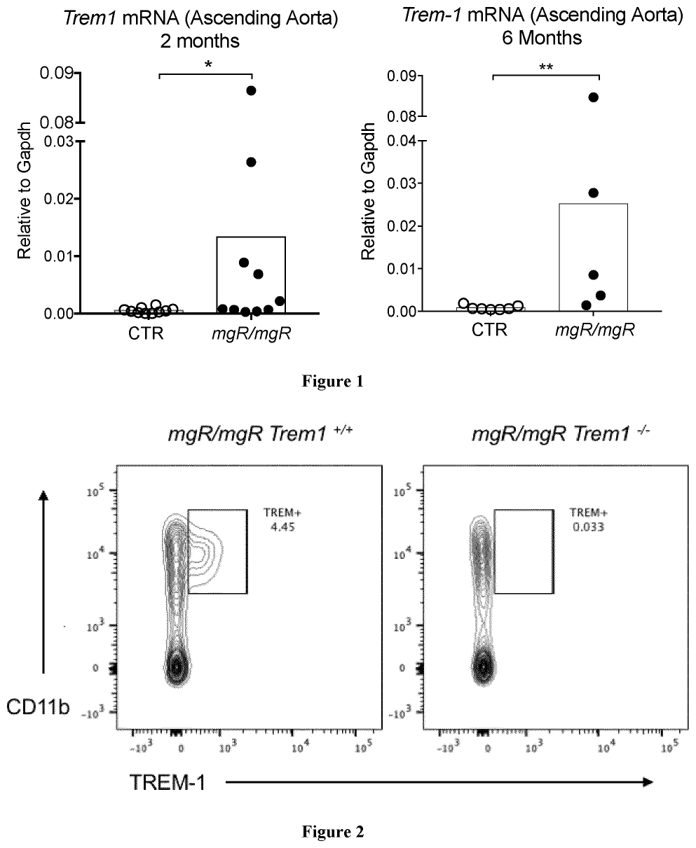

Next, we analyzed by qPCR the gene expression of TREM-1 in the aorta of fibrillin-1 hypomorphic mgR/mgR mice. This is a mouse model of haploinsufficiency that mimics Marfan syndrome and we compared it to littermatte control mice (CTR). We showed that Trem-1 m RNA levels are higher in the aorta of mGr/mGr mice compared to control mice at different times (2 months and 6 months of age) (FIG. 1).

### TREM-1 Deficiency Protects Against Aortic Rupture in a Mouse Model of Marfan Syndrome.

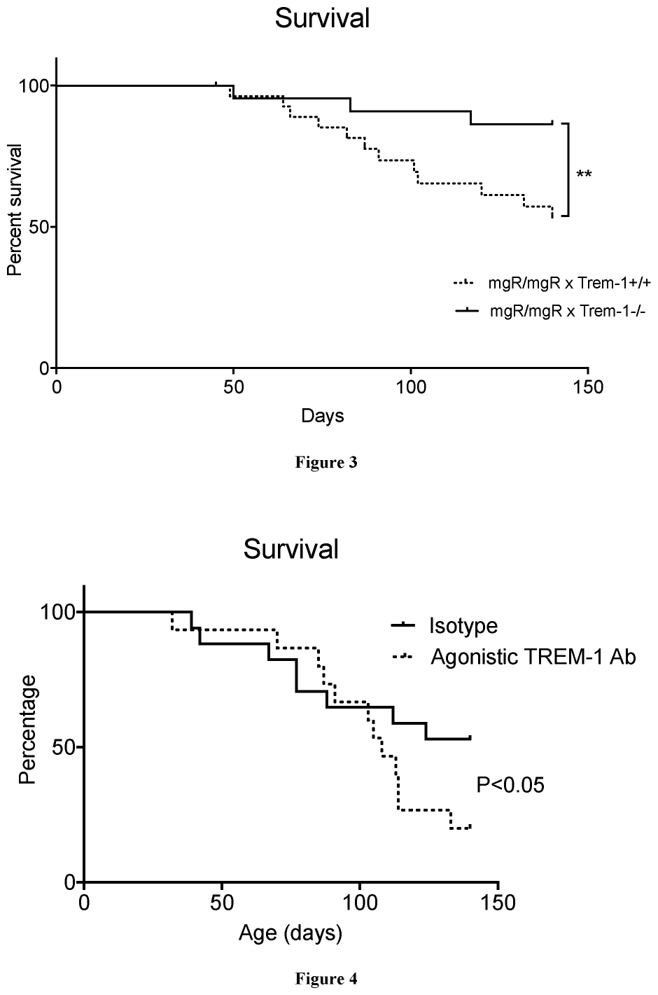

In order to study the role of TREM-1 in the aortic pathology of Marfan syndrome, we crossed mgR/mgR mice (Pereira et al. PNAS 1999) with Trem-1 deficient mice (Trem-1^{-/-}) (Zysset, Nat Commun 2016). Trem-1 deficiency in mgR/mgR/Trem-1^{-/-} mice was confirmed by qPCR and flow cytometry (FIG. 2). The animals (males and females) were followed during a 5-month period. As previously reported, Fibrillin-1 deficiency is responsible for significant mortality by aortic rupture (50%, 14/28) at 5 months. However, we found for the time that Trem-1 deficiency protects against aortic rupture and significantly improves the survival of the mice (12%, 3/26, P=0.004) (FIG. 3).

### In Vivo Overstimulation of TREM-1 Aggravates Aorta Rupture in Marfan Mice

Our loss-of-function experiment supports a pathogenic role of TREM-1 in the complications of Marfan aortopathy in mice.

Next, we evaluated an additional “gain-of-function approach based on in vivo TREM-1 stimulation with agonistic anti-TREM-1 antibody. Six-week old mGR/mgR mice were treated either with isotype (N=15) or with anti-TREM-1 stimulating antibody IP (0.1 mg/kg 2 times/week, 16-0621-86, clone MEL-14) (N=15). We found that TREM-1 overactivation aggravates aorta rupture and mice death supporting a key role of TREM-1 in the pathophysiology of aorta rupture in Marfan syndrome (FIG. 4)

### CONCLUSION

For the first time, we have demonstrated a critical role for TREM-1 in the pathophysiology of ascending aortopathy related to Marfan disease. Deletion of TREM-1 protects against aortic rupture. Blocking TREM-1 could therefore be a therapeutic approach for Marfan-related aortopathy.

### REFERENCES

Throughout this application, various references describe the state of the art to which this invention pertains. The disclosures of these references are hereby incorporated by reference into the present disclosure.

1. A method of treating Marfan Syndrome in a patient in need thereof comprising administering to the patient a therapeutically effective amount of a TREM-1 inhibitor.

2. The method of claim 1 wherein the TREM-1 inhibitor is prevents ascending aorta rupture.

3. The method of claim 1 wherein the TREM-1 inhibitor is an antibody directed to TREM-1.

4. The method of claim 1 wherein the TREM-1 inhibitor is a peptide.

5. The method of claim 4 wherein the TREM-1 inhibitor is a peptide selected from the group consisting of SEQ ID NO:2, SEQ ID NO:3; SEQ ID NO:4, SEQ ID NO:5, SEQ ID NO:6, SEQ ID NO:7, SEQ ID NO:8, SEQ ID NO:9, SEQ ID NO:10 and SEQ ID NO: 11.

6. The method of claim 4 wherein the TREM-1 inhibitor is a short TLT-1 peptide comprising less than 50 amino acids, and wherein said short TLT-1 peptide comprises from 6 to 20, inclusive, consecutive amino acids from human TLT-1 having an amino acid sequence as set forth in SEQ ID NO: 12; or a sequence having at least 60, 65, 70, 75, 80, 85 or 90% identity with the amino acid sequence as set forth in SEQ ID NO: 12; or a function-conservative variant or derivative thereof.

7. The method of claim 4 wherein the TREM-1 inhibitor is a TLT-1 peptide consisting of 6 to 12, 13, 14, 15, 16, 17, 18, 19 or 20 amino acids and comprising an amino acid sequence as set forth in SEQ ID NO: 2, SEQ ID NO: 3, SEQ ID NO: 4, SEQ ID NO: 5 or SEQ ID NO: 6. or a sequence having at least 60, 65, 70, 75, 80, 85 or 90% identity with the amino acid sequence as set forth in SEQ ID NO: 2, SEQ ID NO: 3, SEQ ID NO: 4, SEQ ID NO: 5, or SEQ ID NO: 6, respectively; or a function-conservative variant or derivative thereof.

8. The method of claim 4 wherein the TREM-1 inhibitor is a TLT-1 peptide comprising or consisting of an amino acid sequence as set forth in SEQ ID NO: 2, SEQ ID NO: 3, SEQ ID NO: 4, SEQ ID NO: 5, or SEQ ID NO: 6; or a sequence having at least 60, 65, 70, 75, 80, 85 or 90% identity with the amino acid sequence as set forth in SEQ ID NO: 2, SEQ ID NO: 3, SEQ ID NO: 4, SEQ ID NO: 5, or SEQ ID NO: 6, respectively; or a function-conservative variant or derivative thereof.

9. The method of claim 4 wherein the TREM-1 inhibitor is a TLT-1 peptide having an amino acid sequence as set forth in SEQ ID NO: 2, SEQ ID NO: 3, SEQ ID NO: 4, SEQ ID NO: 5, or SEQ ID NO: 6; or a sequence having at least 60, 65, 70, 75, 80, 85 or 90% identity with the amino acid sequence as set forth in SEQ ID NO: 2, SEQ ID NO: 3, SEQ ID NO: 4, SEQ ID NO: 5, or SEQ ID NO: 6, respectively; or a function-conservative variant or derivative thereof.

10. The method of claim 4 wherein the TREM-1 inhibitor is a TLT-1 peptide having an amino acid sequence as set forth in SEQ ID NO: 3; or a sequence having at least 60, 65, 70, 75, 80, 85 or 90% identity with the amino acid sequence as set forth in SEQ ID NO: 3; or a function-conservative variant or derivative of SEQ ID NO: 3.

11. The method of claim 4 wherein the TREM-1 inhibitor is a short TREM-1 peptide consisting of less than 50 amino acids, and wherein said TREM-1 peptide comprises between 6 and 20 consecutive amino acids from human TREM-1 having an amino acid sequence as set forth in SEQ ID NO: 1 or a function-conservative variant or derivative thereof.

12. The method of claim 4 wherein the TREM-1 inhibitor is a TREM-1 peptide consisting of 6 to 12, 13, 14, 15, 16, 17, 18, 19 or 20 amino acids and comprising an amino acid sequence as set forth in SEQ ID NO: 7, SEQ ID NO: 8, SEQ ID NO: 9, SEQ ID NO: 10 or SEQ ID NO: 11; or a sequence having at least 60, 65, 70, 75, 80, 85 or 90% identity with the amino acid sequence as set forth in SEQ ID NO: 7, SEQ ID NO: 8, SEQ ID NO: 9, SEQ ID NO: 10 or SEQ ID NO: 11, respectively; or a function-conservative variant or derivative thereof.

13. The method of claim 4 wherein the TREM-1 inhibitor is a TREM-1 peptide comprising or consisting of an amino acid sequence as set forth in SEQ ID NO: 7, SEQ ID NO: 8, SEQ ID NO: 9, SEQ ID NO: 10 or SEQ ID NO: 11; or a sequence having at least 60, 65, 70, 75, 80, 85 or 90% identity with the amino acid sequence as set forth in SEQ ID NO: 7, SEQ ID NO: 8, SEQ ID NO: 9, SEQ ID NO: 10 or SEQ ID NO: 11, respectively; or a function-conservative variant or derivative thereof.

14. The method of claim 4 wherein the TREM-1 inhibitor is a TREM-1 peptide having an amino acid sequence as set forth in SEQ ID NO: 7, SEQ ID NO: 8, SEQ ID NO: 9, SEQ ID NO: 10 or SEQ ID NO: 11 or a sequence having at least 60, 65, 70, 75, 80, 85 or 90% identity with the amino acid sequence as set forth in SEQ ID NO: 7, SEQ ID NO: 8, SEQ ID NO: 9, SEQ ID NO: 10 or SEQ ID NO: 11, respectively; or a function-conservative variant or derivative thereof.

15. The method of claim 6 wherein the short TLT-1 peptide comprises from 6 to 20, inclusive, amino acids, or from 6 to 17, inclusive, amino acids.

16. The method of claim 11 wherein the TREM-1 inhibitor is a short TREM-1 peptide having from 6 to 17, inclusive, amino acids.


SEQUENCE LISTING (12 sequences)

SEQ ID NO: 1 - protein, 234 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 2 - protein, 17 aa, Artificial
  Synthetic LR17
LQEEDAGEYGCMVDGAR

SEQ ID NO: 3 - protein, 12 aa, Artificial
  Synthetic LR12
LQEEDAGEYGCM

SEQ ID NO: 4 - protein, 6 aa, Artificial
  Synthetic LR6-1
LQEEDA

SEQ ID NO: 5 - protein, 6 aa, Artificial
  Synthetic LR6-2
EDAGEY

SEQ ID NO: 6 - protein, 6 aa, Artificial
  Synthetic LR6-3
GEYGCM

SEQ ID NO: 7 - protein, 17 aa, Artificial
  Synthetic LP17
LQVEDSGLYQCVIQHPP

SEQ ID NO: 8 - protein, 12 aa, Artificial
  Synthetic LP12
LQVEDSGLYQCV

SEQ ID NO: 9 - protein, 6 aa, Artificial
  Synthetic LP6-1
LQVEDS

SEQ ID NO: 10 - protein, 6 aa, Artificial
  Synthetic LP6-2
EDSGLY

SEQ ID NO: 11 - protein, 6 aa, Artificial
  Synthetic LP6-3
GLYQCV

SEQ ID NO: 12 - protein, 311 aa, Homo sapiens
MGLTLLLLLLLGLEGQGIVGSLPEVLQAPVGSSILVQCHYRLQDVKAQKVWCRFLPEGCQPLVSSAVDRRAPAGRRTFLTDLGGGLLQVEMVTLQEEDAGEYGCMVDGARGPQILHRVSLNILPPEEEEETHKIGSLAENAFSDPAGSANPLEPSQDEKSIPLIWGAVLLVGLLVAAVVLFAVMAKRKQGNRLGVCGRFLSSRVSGMNPSSVVHHVSDSGPAAELPLDVPHIRLDSPPSFDNTTYTSLPLDSPSGKPSLPAPSSLPPLPPKVLVCSKPVTYATVIFPGGNKGGGTSCGPAQNPPNNQTPSS